(19) United States
(12) Patent Application Publication
(10) Pub. No.: US 2021/0275352 A1
(43) Pub. Date: Sep. 9, 2021

(54) LASER SYSTEM FOR PERFORMING OPHTHALMOLOGICAL PROCEDURES

(71) Applicant: FRITZ RUCK OPHTHALMOLOGISCHE SYSTEME GMBH, Eschweiler (DE)

(72) Inventor: Markus PERPEET, Eschweiler (DE)

(21) Appl. No.: 16/328,378

(22) PCT Filed: Aug. 22, 2017
(86) PCT No.: PCT/EP2017/071085

§ 371 Date: Feb. 26, 2019

(30) Foreign Application Priority Data
Aug. 26, 2016 (EP) 16185835.2

(51) Int. Cl. A61F 9/008

(52) U.S. Cl. CPC A61F 9/008; A61B 2018/00761

(57) ABSTRACT

A laser system for performing ophthalmologic procedures comprising a pulsed laser for emitting laser pulses, a focusing optics for generating at least one focal point in the anterior region of a patient's eye, a deflection device for varying the position of the focal point in the anterior region of the patient's eye, and a control unit for controlling the deflection device. The pulsed laser has a pulse duration in the range of nanoseconds, and the laser system is designed as a handheld device.

A laser system for performing ophthalmologic procedures comprising a pulsed laser for emitting laser pulses, a focusing optics for generating at least one focal point in the anterior region of a patient's eye, a deflection device for varying the position of the focal point in the anterior region of the patient's eye, and a control device for controlling the deflection device.

From the publication WO 2011/059958 A2, a laser system for performing ophthalmologic procedures is known, which laser system comprises a focusing optics, a deflection device and a pulsed laser for emitting laser pulses, wherein the pulsed laser is designed for emitting laser pulses with a pulse duration in the range of femtoseconds. Such pulsed lasers are also referred to as femto-pulsed lasers. According to the publication WO 2011/059958 A2, the use of femto-pulsed lasers in ophthalmologic procedures has led to all kinds of advantages over laser systems comprising pulsed lasers for performing ophthalmologic procedures which emit laser pulses with a pulse duration in the range of nano- or picoseconds. Such lasers are also referred to as nano-pulsed lasers or pico-pulsed lasers, respectively. Due to the shorter pulse duration of the laser pulses of a femto-pulsed laser, the energy input into the tissue is reduced, whereby precision and controllability of the procedure is improved and, in consequence thereof, the risk of inadvertent damage to the tissue is minimized. As a result, the use of femto-pulsed lasers has become established in ophthalmology.

However, when femto-pulsed lasers are used, it has turned out to be disadvantageous that, due to the low energy input into the tissue, an exposure time for the tissue is increased, whereby the duration of surgical procedures on the eye is longer.

Moreover, with laser systems known from the prior art, the drawback is obtained that they are very unwieldy to use, since a laser of the laser system is arranged externally to the treatment device that is placed close to the eye during the procedure and the laser and the treatment device are coupled to each other via a flexible optical conductor. Furthermore, because of the flexible optical conductor, the drawback is obtained that the laser system is very sensitive. For example, if the fibre-optic light guide is bent, it must be replaced immediately, since otherwise a defined delivery of the laser light into a tissue of the eye is no longer ensured. Such a laser system comprising a treatment device formed by a handpiece is known, for example, from WO 2014/144697 A1.

It is the object of the present invention to provide a laser system for ophthalmologic procedures, wherein the duration of the procedure on the patient is reduced and which is quick and easy to handle.

The object is achieved according to the invention in that the laser system is configured in a housing in order to form a handheld device, with the housing comprising handles and support points, which support points rest on the nose of the patient during the ophthalmologic procedure.

By using a pulsed laser with a pulse duration in the range of nanoseconds, the prejudice among experts concerning the present invention is dispelled that pulsed lasers which emit laser pulses with a pulse duration in the range of nanoseconds are poorly or not at all usable in ophthalmologic procedures. As a result of the longer pulse duration, the energy input into the tissue increases, whereby procedures on the eye can be performed faster using a nano-pulsed laser, as opposed to a femto-pulsed laser. For example, a femto-pulsed laser requires four seconds for a cut in the anterior region of the eye, whereas a nano-pulsed laser requires only milliseconds. This not only saves time and thus renders the procedure more bearable for the patient, but also makes it possible to design the laser system as a handheld device, since systems that fix the eye or systems that continuously track and scan the eye can be omitted. Fixing the eye during the procedure involves a risk that is not insignificant, since the fixing of the eye may lead to a distortion of the eye, whereby complications may be caused during the lasing of the tissue or, in the worst case, the surgical procedure could have a poor result. By tracking or, respectively, scanning the eye, this disadvantage is indeed avoided, but such systems are still very expensive.

In addition, by using a nano-pulsed laser, the advantage is obtained that the laser system is less expensive than a laser system with a femto-pulsed laser.

By configuring the laser system within a housing and, concomitantly, reducing the size of the laser system, the laser system can be designed as a handheld device. In this connection, a handheld device is considered to be especially a device which is held by a person in one hand, advantageously in both hands, during use. The handheld device is characterized primarily by the fact that it has a compact and lightweight design and can be easily held and carried by an average adult person. Moreover, the handheld device is characterized by being easy to handle and easy to operate due to its ergonomics when the device is being held. The handheld device is portable.

Such a laser system is advantageously used for performing an anterior capsulotomy or a posterior capsulotomy on the freely movable eye in the anterior part of the eye. Due to the design of the laser system as a handheld device, the advantage is thereby obtained that the capsulotomy as well as other procedures on the eye can be performed in the same surgical area or operating room, respectively. Thus, for example, a fragmentation of the ocular lens following the capsulotomy can be effected with the same laser system or else by phacoemulsification. A relocation of the patient between two surgical steps is completely avoided because of the simple handling and the compactness of the handheld device, whereby the possibility of complications is reduced and the procedure is rendered even more bearable for the patient.

Preferably, the pulsed laser comprises a device which is designed for varying a frequency of the laser pulses in order to perform operations also on the posterior part of the eye. This device may comprise mechanical and/or electrical components. As a result, the advantage is obtained that additional equipment can be omitted and a great number of different procedures can be performed with one device.

In a further embodiment variant, the nano-pulsed laser is accommodated in a device arranged externally to the handheld device, wherein the laser pulses are transmitted into the handheld device via fibre optics, e.g., optical fibres.

Further advantageous embodiment variants of the laser system according to the invention for performing ophthalmologic procedures will be explained in more detail hereinbelow with reference to the figures.

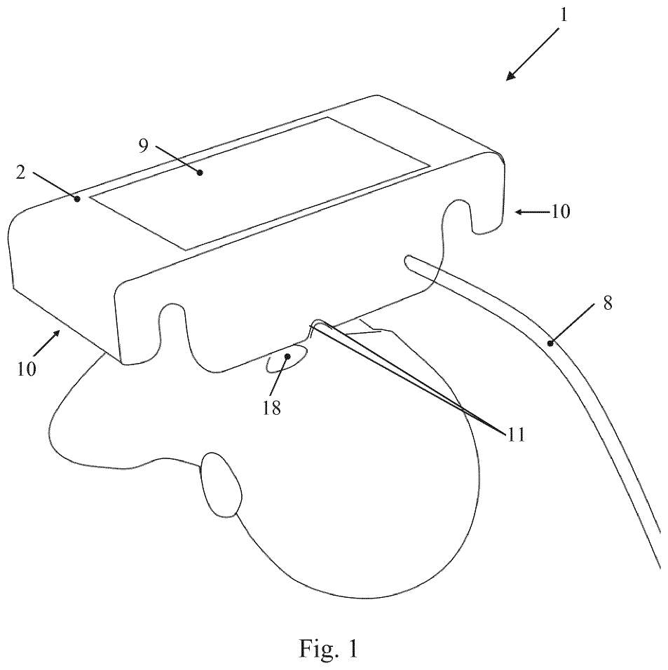

FIG. 1 shows an embodiment variant of a laser system according to the invention in a perspective view.

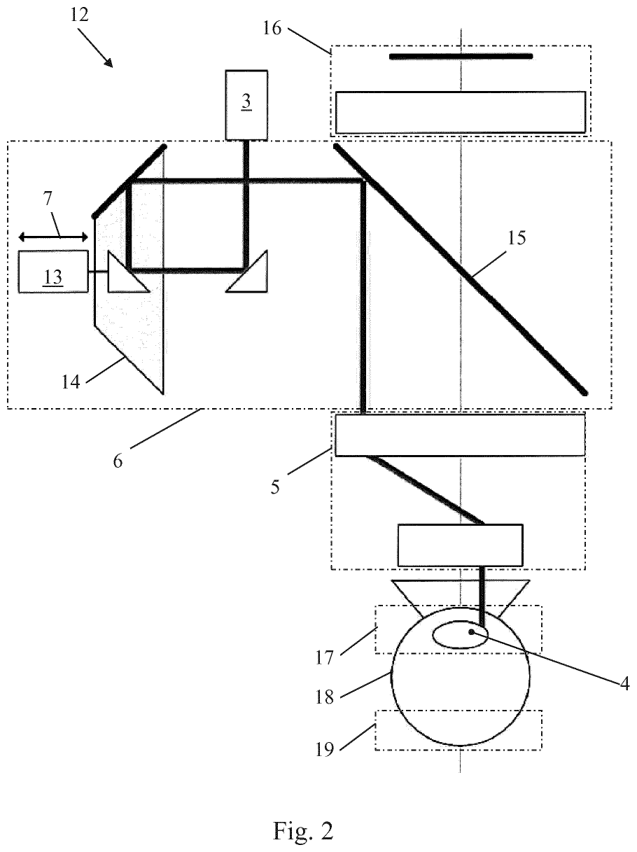

FIG. 2 shows a beam path of the laser system according to FIG. 1 in a schematic view.

FIG. 1 shows an embodiment variant of a laser system 1 according to the invention installed in a housing 2 during the implementation of an ophthalmologic procedure on one eye 18 or both eyes of a patient. The laser system 1 is designed as a handheld device and can be plugged into a power socket by means of a cable 8 for powering the laser system 1. The laser system 1 comprises a nano-pulsed laser 3, a focusing optics 5, a deflection device 6 and a control unit, which is not illustrated. The nano-pulsed laser 3, the focusing optics 5 and the deflection device 6 are shown schematically in FIG. 2. In addition, the laser system 1 comprises a touchscreen 9, lighting means, which are not illustrated, handles 10 and support points 11 for placing the handheld device on the patient.

FIG. 2 shows a beam path 12 of the laser system 1 according to FIG. 1 in a schematic view. A laser pulse generated by the nano-pulsed laser 3 is focused on a focal point on the tissue to be operated on via the deflection device 6 and the focusing optics 5, wherein a position of the focal point on the tissue to be operated on can be varied by means of the deflection device 6. For this purpose, the deflection device 6 has an electric motor 13 the position of which relative to a frustoconical mirror 14 is alterable along the double arrow 7 by means of a linear actuator, which is not illustrated. The nano-pulsed laser 3 is designed for performing surgical procedures on the anterior region 17 of the eye 18, wherein the nano-pulsed laser 3 is advantageously designed for emitting laser pulses in the infrared range. Furthermore, the deflection device 6 comprises a dichroic mirror 15, and the laser system 1 has an image sensor 16. The nano-pulsed laser 3, the electric motor 13, the linear actuator, the lighting means and the touchscreen 9 are connected for communication by means of a control unit, which is not illustrated. Suitably, the lighting means are formed by LEDs and are oriented with respect to the eye 18 in such a way that the eye 18 is located in the light cone of the lighting means.

Subsequently, the implementation of an ophthalmologic procedure with the laser system 1 according to FIG. 1 will be described in more detail. After the patient has assumed an operating position which, in particular, is horizontal, the handheld device is placed on the face of the patient by a surgeon, with the handheld device supported on the face, in particular on the nose of the patient, via the support points 11. For this, the support points 11 are advantageously formed of a soft material, e.g., an elastomer. On the touchscreen 9, the surgeon can choose which operation is carried out. For example, an anterior capsulotomy or a posterior capsulotomy can be performed with the handheld device. In the present case, the surgeon chooses, for example, an anterior capsulotomy. By means of the image sensor 16, the surgeon can orient the focusing optics 5 precisely above the eye 18 of the patient via the touchscreen 9. By activating a switch mounted on the housing 2, which switch is not illustrated, or by choosing an icon on the touch display 9, the capsulotomy is started, wherein fixing, tracking and/or scanning of the eye 18 can be omitted due to the high energy input of the nano-laser 3 and, resulting therefrom, a short exposure time for the tissue of the capsular sac of the eye 18. After the capsule sac has been opened, the handheld device can be removed and set aside. The actual operation on the eye 18 can now be started. For example, the implementation of a fragmentation of a lens 4 by means of phacoemulsification.

In a further embodiment variant, the laser system 1 is designed for fragmenting the lens 4 of the eye 18. As a result, the advantage is obtained that two surgical steps can be performed with the same device, and the duration of the procedure is again minimized.

In a further embodiment variant, the pulse frequency of the laser pulses can be modulated. As a result, the advantage is obtained that procedures on the posterior region 19 of the eye 18 can be carried out as well.

In a further embodiment variant, the laser system 1 has a battery for power supply, which is accommodated in the housing 2.

1. A laser system for performing an ophthalmologic procedure, the laser system comprising:
a pulsed laser for emitting laser pulses, the pulsed laser having a pulse duration in a range of nanoseconds,
a focusing optics for generating at least one focal point in an anterior region of a patient's eye,
a deflection device for varying a position of the focal point in the anterior region of the patient's eye, and
a control unit for controlling the deflection device,
wherein the laser system is configured in a housing of the laser system in order to form a handheld device, the housing comprising handles and support points, wherein the support points are configured to rest on a face and on nose of the patient during the ophthalmologic procedure.

2. A laser system according to claim 1, wherein the laser system is designed for performing an anterior capsulotomy or a posterior capsulotomy on the freely movable eye.

3. A laser system according to claim 2, wherein the pulsed laser comprises a device which is designed for varying a frequency of the laser pulses in order to perform operations on the posterior region of the eye.

4. A laser system according to claim 1, wherein the laser system is designed for a fragmentation of a lens of the eye.

5. A laser system according to claim 1, wherein that the laser system comprises a touchscreen.

6. A laser system according to claim 1, wherein the laser system has at least one lighting means designed for illuminating the eye during the ophthalmologic procedure.

7. A laser system according to claim 1, wherein the laser system has a cable for power supply, via which the laser system can be plugged into a power socket.
